(19) United States
(12) Patent Application Publication
(10) Pub. No.: US 2015/0321985 A1
(43) Pub. Date: Nov. 12, 2015

(54) (HYDROXYSTILBENE COMPOUND)-(SINAPINIC ACID) REACTION PRODUCT HAVING ANTIFEEDING ACTIVITY

(71) Applicants: UHA MIKAKUTO CO., LTD., Yamatokooriyama-shi, Nara (JP); NATIONAL UNIVERSITY CORPORATION SHIZUOKA UNIVERSITY, Shizuoka-shi, Shizuoka (JP)

(72) Inventors: Satoshi DOI, Yamatokooriyama-shi (JP); Akinobu KISHI, Yamatokooriyama-shi (JP); Taiji MATSUKAWA, Yamatokooriyama-shi (JP); Takeki MATSUI, Yamatokooriyama-shi (JP); Yasumasa YAMADA, Yamatokooriyama-shi (JP); Ichiro YAMADA, Yamatokooriyama-shi (JP); Kazutoshi SAYAMA, Shizuoka-shi (JP)

(21) Appl. No.: 14/655,124

(22) PCT Filed: Dec. 20, 2013
(86) PCT No.: PCT/JP2013/084309

§ 371 Date: Jun. 24, 2015

(30) Foreign Application Priority Data
Dec. 25, 2012 (JP) 2012-280888

(51) Int. Cl. C07C 43/23

(52) U.S. Cl. CPC C07C 43/23

(57) ABSTRACT

Provided is an antifeeding agent including a (hydroxystilbene compound)-(sinapinic acid) reaction product represented by Formula (1) (in Formula (1), each of R₁ to R₄ is a hydrogen atom, a hydroxy group, a saturated or unsaturated, straight or branched chain alkoxy group having 1 to 10 carbon atoms, or a saturated or unsaturated, straight or branched chain alkyl group having 1 to 10 carbon atoms; and R₁ to R₄ are optionally the same as or different from each other) or a pharmaceutically acceptable salt thereof. The antifeeding agent is a novel, safe, highly practical antifeeding agent that can be taken for a long period of time.

### TECHNICAL FIELD

The present invention relates to an antifeeding agent including a (hydroxystilbene compound)-(sinapinic acid) reaction product that is obtained by a very simple synthetic method applicable to foods, as the active component.

### BACKGROUND ART

At the present time, a large number of compounds are prepared by fermentation, extraction, organic synthesis, and various other techniques and are used in a wide variety of fields such as foods and pharmaceutical products. Among them, only a few compounds satisfy both usability and practicality. In addition, novel compounds that have not been reported are more difficult to satisfy both usability and practicality due to the cost of raw materials for synthesis or the difficulty of isolation technique.

The inventors of the present invention have found a revolutionary method for synthesizing a novel compound by using a component derived from food, and the method is simple and enables easy isolation of the compound (Patent Document 1 and Patent Document 2). However, the method is applied to limited fields, and is supposed to have a large number of revolutionary, unknown applications.

In modern social life, excess stress, excess food intake, and insufficient exercise are rampant among people and cause metabolic syndrome, which becomes a social problem. The metabolic syndrome is a condition of visceral fat accumulation in combination of two or more of hyperglycemia, high blood pressure, and dyslipidemia and is a condition where the risk of cardiovascular diseases such as cerebral infarction, arteriosclerosis, and myocardial infarction is increased.

To prevent the metabolic syndrome, moderate exercise and appropriate energy intake, or the control of excess food intake, are most effective. However, in developed countries where a lot of food is available and easily taken, such as modern Japan, it is difficult to suppress appetite and to spontaneously control excess food intake in many cases.

In such circumstances, pharmaceutical drugs of suppressing appetite have been developed. For example, an amphetamine, mazindol, is put into practical use as an anti-obesity agent having antifeeding activity. The drug unfortunately has a high risk of side effects and the like and thus is limitedly used only for severe obesity patients. Although obesity needs to be prevented or treated for a long period of time, orlistat is the only anti-obesity agent usable for a long period of time, at the present time.

To address such circumstances, new antifeeding agents have been being developed for the prevention or treatment of obesity. In 1998, it was revealed that melanin-concentrating hormone (MCH) has an important role in feeding behavior, for example (Non-Patent Document 1). Non-Patent Document 1 reports that the food intake is decreased and the weight is reduced in a MCH-knockout mouse. In addition, an orphan G-protein-coupled receptor (GPCR), MCH1R, was identified as the receptor of MCH (Non-Patent Documents 2 and 3). These findings suggest that an MCH antagonist is useful as the antifeeding agent, and many MCH antagonists are reported. For example, an MCH antagonist containing a thienopyrimidone compound as the active component is reported (Patent Document 3). Other MCH antagonists are also reported, but no effect of such MCH antagonists on animals or human beings is shown, and it is unknown whether the antagonists exhibit the antifeeding activity in animals and human beings in practice.

Separately, a neuropeptide, neuropeptide Y (NPY), is known to have strong hyperphagia activity. The receptor of NPY is a G-protein-coupled receptor, and six subtypes from Yl to y6 are identified. An NPYY5 antagonist is reported as an antagonist of these receptors (Patent Document 4). However, Patent Document 4 merely evaluates the antagonistic action against the receptor, and it is unknown whether the antagonist exhibits the antifeeding activity in animals and human beings in practice.

The inventions described in Patent Documents above are antifeeding agents focused on pharmaceutical products, whereas antifeeding agents usable as foods are also disclosed. The agents disclosed are exemplified by a central nervous function improver including a peptide derived from rapeseed as the active component (Patent Document 5), a peptide-containing antifeeding agent including MCH derived from fish and a peptide thereof as the active component (Patent Document 6), an antifeeding agent including an extract derived from avocado as the active component (Patent Document 7), and an antifeeding agent including chitobiose and chitotriose as the active components (Patent Document 8). These antifeeding agents exhibit the antifeeding activity on animals in the examples, but the activity was evaluated only by the food intake immediately after administration, and the effect on long-term feeding behavior is not examined. Patent Document 8 also discloses the result of conversely inducing the feeding behavior after fasting, and this result is unfavorable as the antifeeding agent. In such circumstances, promising agents are hydroxystilbenes including resveratrol.

Hydroxystilbenes contained in various plant species have been reported to have various physiological activities. For example, resveratrol, which is well-known as a component in red wine, is a hydroxystilbene contained in grape peel and the like, and plants are considered to contain the resveratrol as a phytoalexin for the protection of themselves from pathogens or for the protection from UV light. In 1997, high anticancer activity of resveratrol was reported (Non-Patent Document 4). This report has brought attention to physiological activities of hydroxystilbenes including resveratrol, and it is being revealed that such hydroxystilbenes have effects on various diseases (Non-Patent Document 5). In addition, it is known that “French paradox”, which is a phenomenon of extremely low incidence rate of cardiovascular diseases in France, has a correlation with the intake amount of red wine and is considered to be due to cardiovascular protective effect by antioxidant activity, antiinflammatory activity, and similar activities of the resveratrol. On this account, hydroxystilbenes including resveratrol have drawn attention as a functional component having both efficacy and safety.

Resveratrol is suggested to have antifeeding activity by suppressing the expression of agouti-related peptide (AgRP) and NPY (Non-Patent Document 6). Non-Patent Document 6 reports that administration of 100 mg/kg of resveratrol significantly reduced the diet intake and resveratrol suppressed the expression of AgRP and NPY by means of reporter assay of AgRP and NPY in cultured cells. It is also suggested that resveratrol passes through the blood-brain barrier and directly affects neurons and the like, and thus the resveratrol is expected as a new antifeeding agent. As described later in examples, even when administered for a long period of time, resveratrol unfortunately exhibits very low antifeeding activity, and the antifeeding activity of the resveratrol itself is insufficient.

On this account, there is a demand for development of a novel, safe and practical antifeeding agent.

### CITATION LIST

### Patent Literature

Patent Document 1: JP-A No. 2012-246243
Patent Document 2: International Publication WO 2012/070656
Patent Document 3: Japanese Patent No. 5000490
Patent Document 4: Japanese Patent No. 3910446
Patent Document 5: Japanese Patent No. 4570402
Patent Document 6: Japanese Patent No. 4593639
Patent Document 7: Japanese Patent No. 4693964
Patent Document 8: Japanese Patent No. 3836894

### Non-Patent Literature

Non-Patent Document 1: Nature, 396, pp. 670-674 (1998)
Non-Patent Document 2: Nature, 400, pp. 265-269 (1999)
Non-Patent Document 3: Tread. Endocri. Metab., 11, pp. 299-303 (2000)
Non-Patent Document 4: Science, 275 (10), pp. 218-220 (1997)
Non-Patent Document 5: Drug Discovery, 5, pp. 493-506 (2006)
Non-Patent Document 6: Arch. Pharm. Res., 33 (5), pp. 775-780 (2010)

### SUMMARY OF INVENTION

### Technical Problem

As described above, the inventors of the present invention have studied to enhance the functionality of functional materials including hydroxystilbenes, and as an example, have achieved the synthesis of a large number of novel hydroxystilbene-cinnamic acid reaction products by heat treatment of hydroxystilbenes and cinnamic acids at high temperature or under high pressure (Patent Document 1 and Patent Document 2). The synthetic method of these novel hydroxystilbene-cinnamic acid reaction products is simple and excellent because it is applicable to food, but the functionality of the reaction products have been examined insufficiently. On this account, the inventors of the present invention have studied the functionality of these novel (hydroxystilbene compound)-(sinapinic acid) reaction products, have found that a certain (hydroxystilbene compound)-(sinapinic acid) reaction product has antifeeding activity, and have accomplished the present invention.

Hence, the present invention has an object to provide a novel, safe, highly practical antifeeding agent that can be taken for a long period of time.

### Solution to Problem

The summary of the present invention is as follows:

[1] an antifeeding agent including a (hydroxystilbene compound)-(sinapinic acid) reaction product represented by Formula (1):

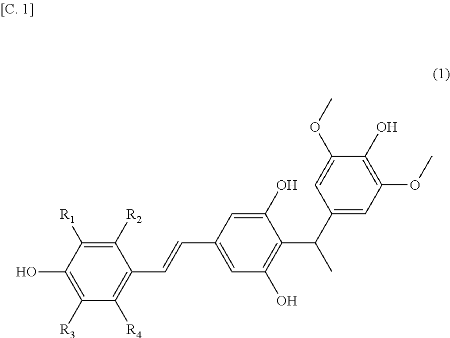

(in Formula (1), each of R₁ to R₄ is a hydrogen atom, a hydroxy group, a saturated or unsaturated, straight or branched chain alkoxy group having 1 to 10 carbon atoms, or a saturated or unsaturated, straight or branched chain alkyl group having 1 to 10 carbon atoms; and R₁ to R₄ are optionally the same as or different from each other)
or a pharmaceutically acceptable salt thereof; and
[2] the antifeeding agent according to the aspect [1], in which R₁ to R₄ are a hydrogen atom in Formula (1).

### Advantageous Effects of Invention

The antifeeding agent of the present invention is highly safe, can be taken for a long period of time to achieve high practicality, and thus is useful as a novel antifeeding agent.

### BRIEF DESCRIPTION OF THE DRAWINGS

FIG. 1 shows results of HPLC analysis carried out in Example 1. The upper chromatogram is the result before reaction, and the lower chromatogram is the result after the reaction. “F” indicates the peak of a (hydroxystilbene compound)-(sinapinic acid) reaction product produced from trans-resveratrol and sinapinic acid as raw materials.

FIG. 2 is a graph showing the change in diet intake of mice in Example 2. The diet intake is a mean value of the weight of diet that a mouse has taken for a week. In the drawing, “ND” represents a normal diet group; “HFD” represents a high-fat diet group; “HFD+0.4% RSV” represents a resveratrol intake group (0.4%); and “HFD+0.05% RD” represents a UHA1028 intake group (0.05%). “a” indicates a significant difference (p<0.05) from the normal diet group in the t test.

FIG. 3 is a graph showing the change in weight increase of mice in Example 2. In the drawing, “ND” represents a normal diet group; “HFD” represents a high-fat diet group; “HFD+0.4% RSV” represents a resveratrol intake group (0.4%); and “HFD+0.4% RD” represents a UHA1028 intake group (0.4%). “a” indicates a significant difference (p<0.05) from the normal diet group in the t test; and “b” indicates a significant difference (p<0.05) from the high-fat diet group in the t test.

### DESCRIPTION OF EMBODIMENTS

The present invention will be described in detail hereinbelow.

The antifeeding agent of the present invention is characterized by including a (hydroxystilbene compound)-(sinapinic acid) reaction product represented by Formula (1):

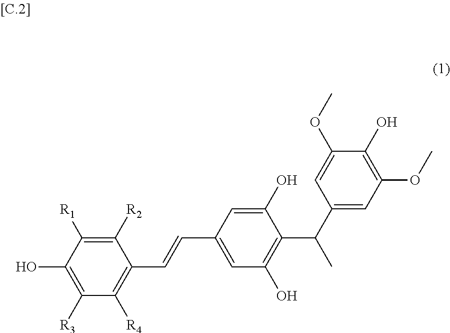

(in Formula (1), each of R₁ to R₄ is a hydrogen atom, a hydroxy group, a saturated or unsaturated, straight or branched chain alkoxy group having 1 to 10 carbon atoms, or a saturated or unsaturated, straight or branched chain alkyl group having 1 to 10 carbon atoms; and R₁ to R₄ are optionally the same as or different from each other) or a pharmaceutically acceptable salt thereof.

In Formula (1), the saturated or unsaturated, straight or branched chain alkoxy group having 1 to 10 carbon atoms represented by R₁ to R₄ is not limited to particular groups but is preferably a straight or branched chain alkoxy group having 1 to 4 carbon atoms. Specific examples of the group include a methoxy group, an ethoxy group, an n-propoxy group, an isopropoxy group, an n-butoxy group, an isobutoxy group, an s-butoxy group, and a t-butoxy group.

The saturated or unsaturated, straight or branched chain alkyl group having 1 to 10 carbon atoms represented by R₁ to R₄ is not limited to particular groups but is preferably a straight or branched chain alkyl group having 1 to 5 carbon atoms. Specific examples of the group include a methyl group, an ethyl group, an n-propyl group, an isopropyl group, an n-butyl group, an isobutyl group, an s-butyl group, a t-butyl group, an n-pentyl group, an isopentyl group, a t-pentyl group, and a neopentyl group.

In the (hydroxystilbene compound)-(sinapinic acid) reaction product represented by Formula (1), at least one of R₁ to R₄ is preferably a hydrogen atom. It is more preferred that each of R₁ to R₄ be a hydrogen atom.

In the present invention, the “antifeeding agent” means a pharmaceutical drug capable of reducing the food intake of human beings, non-human animals, and the like. The antifeeding activity in such mammals can be determined specifically by the method described in examples later.

In the (hydroxystilbene compound)-(sinapinic acid) reaction product represented by Formula (1), the carbon-carbon double bond may be a trans-configuration or a cis-configuration. The (hydroxystilbene compound)-(sinapinic acid) reaction product may be a mixture of a cis isomer and a trans isomer.

The hydroxystilbene may be a natural product or may be a chemically synthesized product with high purity. When used, the hydroxystilbene derived from nature is not necessarily completely purified. A mixture containing other components in addition to the hydroxystilbene can be used if an intended formation reaction proceeds to finally yield the composition of the present invention as described later.

However, in order to efficiently yield the (hydroxystilbene compound)-(sinapinic acid) reaction product of the present invention, a certain amount of the hydroxystilbene is required, and thus a mixture containing 1% by weight or more of the hydroxystilbene can be used as the raw material.

Examples of the mixture containing the hydroxystilbene include extracts of raw materials including grape peel, wine, wine concentrated powder, melinjo, lingonberry, peanut, Polygonum cuspidatum root or rhizome, and passion fruit seeds, and freeze dried products of these extracts.

Examples of the pharmaceutically acceptable salt of the (hydroxystilbene compound)-(sinapinic acid) reaction product include alkali metal salts such as a lithium salt, a sodium salt, and a potassium salt; alkaline earth metal salts such as a magnesium salt, a calcium salt, and a barium salt; an aluminum salt; metal hydroxide salts such as an aluminum hydroxide salt; amine salts such as alkylamine salts, dialkylamine salts, trialkylamine salts, alkylenediamine salts, cycloalkylamine salts, arylamine salts, aralkylamine salts, and heterocyclic amine salts; amino acid salts such as α-amino acid salts and ω-amino acid salts; and peptide salts and primary, secondary, tertiary, and quaternary amines derived therefrom. These pharmaceutically acceptable salts may be used singly or as a mixture of two or more of them.

The (hydroxystilbene compound)-(sinapinic acid) reaction product represented by Formula (1) and a pharmaceutically acceptable salt thereof (hereinafter collectively called (hydroxystilbene compound)-(sinapinic acid) reaction product) can be chemically synthesized in accordance with a method well-known in the art, but such a method involves complicated processes and harmful reagents and processes and thus has disadvantages in terms of safety and recovery ratio.

To address these problems, the inventors of the present invention have intensively studied and consequently have found that heat treatment of a hydroxystilbene and sinapinic acid as described in Patent Documents 1 and 2 enables the efficient and safe production of a (hydroxystilbene compound)-(sinapinic acid) reaction product without requiring harmful reagents or processes, which are required in the chemical synthesis above.

The hydroxystilbene used in the present invention is a hydroxystilbene derivative represented by Formula (2):

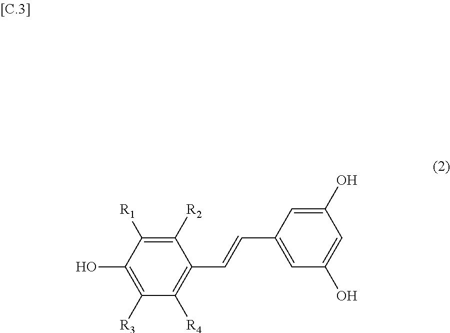

(in Formula (2), R₁ to R₄ are a hydrogen atom, a hydroxy group, a saturated or unsaturated, straight or branched chain alkoxy group having 1 to 10 carbon atoms, or a saturated or unsaturated, straight or branched chain alkyl group having 1 to 10 carbon atoms; and R₁ to R₄ are optionally the same as or different from each other)
and a pharmaceutically acceptable salt thereof.

In Formula (2), the saturated or unsaturated, straight or branched chain alkoxy group having 1 to 10 carbon atoms represented by R₁ to R₄ is not limited to particular groups but is preferably a straight or branched chain alkoxy group having 1 to 4 carbon atoms. Specific examples of the group include a methoxy group, an ethoxy group, an n-propoxy group, an isopropoxy group, an n-butoxy group, an isobutoxy group, an s-butoxy group, and a t-butoxy group.

The saturated or unsaturated, straight or branched chain alkyl group having 1 to 10 carbon atoms represented by R₁ to R₄ is not limited to particular groups but is preferably a straight or branched chain alkyl group having 1 to 5 carbon atoms. Specific examples of the group include a methyl group, an ethyl group, an n-propyl group, an isopropyl group, an n-butyl group, an isobutyl group, an s-butyl group, a t-butyl group, an n-pentyl group, an isopentyl group, a t-pentyl group, and a neopentyl group. Specifically, at least one of R₁ to R₄ is preferably a hydrogen atom, and the compound represented by Formula (2) is more preferably resveratrol or piceatannol.

The compound represented by Formula (2) includes a trans structural isomer and a cis structural isomer, which are partially isomerized to each other by heat or ultraviolet light. In the present invention, the hydroxystilbene may thus be the trans isomer, the cis isomer, or a mixture of the trans isomer and the cis isomer.

Examples of the pharmaceutically acceptable salt of the compound represented by Formula (2) include alkali metal salts such as a lithium salt, a sodium salt, and a potassium salt; alkaline earth metal salts such as a magnesium salt, a calcium salt, and a barium salt; an aluminum salt; metal hydroxide salts such as an aluminum hydroxide salt; amine salts such as alkylamine salts, dialkylamine salts, trialkylamine salts, alkylenediamine salts, cycloalkylamine salts, arylamine salts, aralkylamine salts, and heterocyclic amine salts; amino acid salts such as α-amino acid salts and ω-amino acid salts; and peptide salts and primary, secondary, tertiary, and quaternary amine salts derived therefrom. These pharmaceutically acceptable salts may be used singly or as a mixture of two or more of them.

The sinapinic acid used in the present invention may be a natural product or may be a chemically synthesized product with high purity. When used, the sinapinic acid derived from nature is not necessarily completely purified. A mixture containing other components in addition to the sinapinic acid can be used if an intended formation reaction proceeds to finally yield the (hydroxystilbene compound)-(sinapinic acid) reaction product used in the present invention as described later.

The (hydroxystilbene compound)-(sinapinic acid) reaction product can be produced by the following procedure, for example.

First, a hydroxystilbene and sinapinic acid are dissolved or dispersed in an appropriate solvent. At the time, the solvent is not limited to particular solvents and can be any of water, mixed solutions of water and organic solvents, and organic solvents. If used, the mixed solution of water and an organic solvent may have any blending ratio and may contain any organic solvent. Specifically, a single solvent of water, methanol, or ethanol, a mixed solution of water and methanol, a mixed solution of water and ethanol, or a similar mixed solution is preferably used from the viewpoint of safety and cost.

The obtained solution containing a (hydroxystilbene compound)-(sinapinic acid), or a mixture of a hydroxystilbene and sinapinic acid may have any concentration of the hydroxystilbene and any concentration of the sinapinic acid. However, a solution having a higher hydroxystilbene concentration and/or a higher sinapinic acid concentration advantageously contains a smaller amount of a solvent, and thus the concentrations of the hydroxystilbene and the sinapinic acid are preferably high in corresponding solvents and are more preferably saturated concentrations or higher.

The hydroxystilbene and the sinapinic acid are not necessarily completely dissolved in the solution before the formation reaction. For example, when a solution containing the hydroxystilbene is mixed with a solution containing the sinapinic acid, the concentration of the hydroxystilbene and the concentration of the sinapinic acid in the respective solutions can be the corresponding saturated concentrations or higher.

The pH of a solution containing the hydroxystilbene and the sinapinic acid (hereinafter called (hydroxystilbene compound)-(sinapinic acid)-containing solution) is preferably adjusted to less than 8. For example, a (hydroxystilbene compound)-(sinapinic acid)-containing solution may be prepared, and then a pH adjuster may be added to the solution to adjust the pH. Alternatively, the pH of a solvent may be adjusted before preparation of the solution. The pH at the start of the reaction is preferably not less than 3 and less than 8 from the viewpoint of the recovery ratio of the (hydroxystilbene compound)-(sinapinic acid) reaction product.

Into the (hydroxystilbene compound)-(sinapinic acid)-containing solution, a metal salt is added. The metal salt can be any of acid salts, basic salts, and normal salts and can be any of simple salts, double salts, and complex salts. The metal salts can be a single type or a mixture of a plurality of types. The metal salt is preferably a salt approved as a food additive from the viewpoint of safety. Examples of the salt include magnesium salts, calcium salts, sodium salts, potassium salts, zinc salts, and copper salts that are approved to be added to foods.

Examples of the mixture of metal salts include substances containing several metal salts, such as a mineral mixture mainly containing zinc gluconate, ferric ammonium citrate, calcium lactate, copper gluconate, and magnesium phosphate. The mixture containing a plurality of metal salts is also exemplified by mineral water.

The amount of the metal salt is any amount capable of producing the (hydroxystilbene compound)-(sinapinic acid) reaction product.

Next, in the presence of the metal salt, the (hydroxystilbene compound)-(sinapinic acid)-containing solution is subjected to heat treatment. By the heat treatment, the formation reaction of the (hydroxystilbene compound)-(sinapinic acid) reaction product is carried out. In order to efficiently promote the formation reaction, the heating temperature of the (hydroxystilbene compound)-(sinapinic acid)-containing solution is preferably adjusted to 110° C. or more. In consideration of the boiling point of a solvent used, the solution is preferably heated under pressure. The solution is preferably heated in such a manner that the solution temperature at least partially reaches 110° C. or more. For example, the (hydroxystilbene compound)-(sinapinic acid)-containing solution is placed in an open vessel, and the vessel is heated at a high temperature of higher than the boiling point of the solvent; the (hydroxystilbene compound)-(sinapinic acid)-containing solution is placed in a closed vessel, and the vessel is heated; or the solution is pressurized and heated in a retort apparatus or an autoclave. From the viewpoint of recovery efficiency, the solution temperature more preferably, uniformly reaches 110° C. to 150° C. As with the heating temperature, the heating time is not limited to particular periods of time and can be set to such a period of time that an intended reaction proceeds efficiently. In particular, the heating time varies with the heating temperature and is preferably set in response to the heating temperature. For example, for the heating around 130° C., the heating time is preferably 5 minutes to 480 minutes. The solution may be heated once or may be repeatedly heated a plurality of times. When the solution is heated a plurality of times, a solvent is preferably newly added in order to compensate for a solvent evaporated.

The completion of the formation reaction of the (hydroxystilbene compound)-(sinapinic acid) reaction product by the heat treatment can be ascertained, for example, by HPLC analysis of components in the reaction solution to determine the formation amount of the (hydroxystilbene compound)-(sinapinic acid) reaction product.

The obtained reaction solution may be concentrated or purified. The concentration and the purification can be carried out by a known method. For example, the (hydroxystilbene compound)-(sinapinic acid) reaction product can be concentrated by solvent extraction with chloroform, ethyl acetate, ethanol, methanol, or other solvents, supercritical extraction with carbon dioxide gas, or other extraction techniques. The concentration or the purification can also be carried out by using column chromatography. Recrystallization and membrane treatment such as ultrafiltration membrane treatment are also applicable.

For separation and recovery of the (hydroxystilbene compound)-(sinapinic acid) reaction product from the reaction solution, column chromatography, HPLC, or other separation techniques can be employed.

As needed, the concentrated or purified product may be subjected to vacuum drying or freeze drying to remove solvents, giving a powdery solid.

As needed, the obtained (hydroxystilbene compound)-(sinapinic acid) reaction product may be subjected to a method known in the art to give a salt of the (hydroxystilbene compound)-(sinapinic acid) reaction product.

The (hydroxystilbene compound)-(sinapinic acid) reaction product obtained as above has excellent antifeeding activity, whereas the hydroxystilbene as the raw material lacks such an activity.

The present invention can therefore provide an excellent antifeeding agent that contains the (hydroxystilbene compound)-(sinapinic acid) reaction product as the active component.

The present invention will next be described in detail with reference to examples, but the present invention is not intended to be limited to these examples alone. Here, the reaction using trans-resveratrol as the hydroxystilbene will be described, but a similar reaction using another hydroxystilbene can yield a (hydroxystilbene compound)-(sinapinic acid) reaction product.

### EXAMPLES

### Example 1

### Formation and Isolation of UHA1028

In accordance with the method described in Example 7 in Patent Document 2, trans-resveratrol and sinapinic acid were heated to yield a (hydroxystilbene compound)-(sinapinic acid) reaction product.

In other words, 1 g of trans-resveratrol and 1 g of sinapinic acid (manufactured by Wako Pure Chemical Industries, Ltd.) were dissolved in 20 mL of ethanol, and 20 mL of mineral water was added to give a resveratrol-sinapinic acid-containing solution (pH=4.9). The resveratrol-sinapinic acid-containing solution was heated in an autoclave at 130° C. for 90 minutes. Of the obtained reaction solution, 1 mL was diluted with methanol in a measuring flask to 50 mL and was subjected to HPLC analysis.

The HPLC analysis was performed under the following conditions:

Column: Reversed-phase column “Develosil (registered trademark) C-30-UG-5” (4.6 mm i.d.×250 mm)
Mobile phase: A . . . H₂O (containing 0.1% trifluoroacetic acid (TFA)); B . . . acetonitrile (containing 0.1% TFA)
Flow rate: 1 mL/min

### Injection: 10 μL

### Detection: 254 nm

Gradient (% by volume): From 80% A/20% B to 20% A/80% B for 30 minutes, then from 20% A/80% B to 100% B for 5 minutes, and 100% B for 10 minutes (all linear gradient)

The obtained chromatograms are shown in FIG. 1. The upper chart is the chromatogram before the formation reaction, and the lower chart is the chromatogram after the formation reaction. As shown in the lower chart, the formation of a plurality of compounds was observed including the peak F.

Next, in accordance with the method described in Example 3 in Patent Document 2, the compound contained in the peak F was isolated by preparative HPLC, and the obtained eluate was dried in a usual manner, giving 129 mg of brown powdery substance (UHA1028).

Next, the molecular weight of the UHA1028 was determined by high-resolution electron ionization mass spectrometry, giving a measured value of 408.4436. The measured value was compared with the theoretical value, giving the following molecular formula.

Theoretical value for C₂₄H₂₄O₆ (M+): 408.4438
Molecular formula C₂₄H₂₄O₆

Next, the UHA1028 was subjected to nuclear magnetic resonance (NMR) measurement. The analysis of ¹H-NMR, ¹³C-NMR, and various two-dimensional NMR data revealed that the UHA1028 was a compound represented by Formula (3):

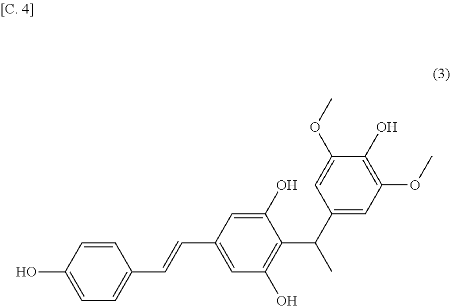 [SMILES: COc1cc(C(C)c2c(O)cc(/C=C/c3ccc(O)cc3)cc2O)cc(OC)c1O]

which corresponds to Formula (1) in which each of R₁, to R₄ is a hydrogen atom.

For the NMR measurement data, the UHA1028 is represented as follows:

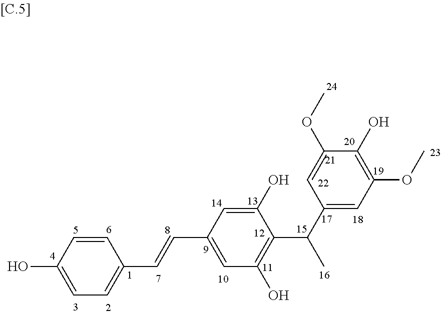 [SMILES: COc1cc(C(C)c2c(O)cc(/C=C/c3ccc(O)cc3)cc2O)cc(OC)c1O]

and the ¹H nuclear magnetic resonance spectrum and the ¹³C nuclear magnetic resonance spectrum are shown in Table 1.

The values are indicated as 6 (ppm), and the UHA1028 was dissolved in methanol-d₃ as the solvent for the measurement.

TABLE 1
NMR Data

| | UHA1028 | |
|---|---|---|
| | 13C | 1H |
| 1 | 130.5 | |
| 2, 6 | 128.6 | 7.32 (2H, d, J = 8.2 Hz) |
| 3, 5 | 116.4 | 6.74 (2H, d, J = 8.2 Hz) |
| 4 | 158.1 | |
| 7 | 128.5 | 6.91 (1H, d, J = 16.0 Hz) |
| 8 | 126.9 | 6.75 (1H, d, J = 16.0 Hz) |
| 9 | 138.0 | |
| 10, 14 | 106.2 | 6.48 (2H, s) |
| 11, 13 | 157.5 | |
| 12 | 120.2 | |
| 15 | 35.1 | 4.68 (1H, d, J = 7.3 Hz) |
| 16 | 18.6 | 1.69 (3H, d, J = 7.3 Hz) |
| 17 | 138.8 | |
| 18, 22 | 106.0 | 6.71 (2H, s) |
| 19, 21 | 148.5 | |
| 20 | 133.9 | |
| 23, 24 | 56.6 | 3.77 (6H, s) |

### Example 2

### Examination of Antifeeding Activity

To examine the antifeeding activity of UHA1028, 8-week-old female mice C57BL/6j (manufactured by CLEA Japan, Inc.) were used. Specifically, the mice were preliminary fed (diet: usual diet) for 7 days, and then were divided into seven groups (a normal diet group, a high-fat diet group, a 0.4% resveratrol intake group, a 0.4% UHA1028 intake group, a 0.2% UHA1028 intake group, a 0.1% UHA1028 intake group, and a 0.05% UHA1028 intake group) each including ten mice in such a manner that each group had substantially the same average weight. The mice were freely fed for 42 days. During the feeding period of test diets, the diet intake and the weight were measured twice a week.

The formulation of each test diet and the amounts of test substances (resveratrol and UHA1028) used in the test are shown in Table 2. A required amount of the UHA1028 was obtained by repeating the formation and isolation described in Example 1.

TABLE 2
Diet formulations and amounts of test substances

| | Normal diet | High-fat diet | Resveratrol | | | | |
|---|---|---|---|---|---|---|---|
| | group | group | intake group | UHA1028 intake group | | | |
| Percentage of test | — | — | 0.4% | 0.4% | 0.2% | 0.1% | 0.05% |
| substance | | | | | | | |
| AIN-76 | 1000 g | 790 g | 790 g | 790 g | 790 g | 790 g | 790 g |
| Lard | | 170 g | 170 g | 170 g | 170 g | 170 g | 170 g |
| Corn oil | | 30 g | 30 g | 30 g | 30 g | 30 g | 30 g |
| Cholesterol | | 10 g | 10 g | 10 g | 10 g | 10 g | 10 g |
| Resveratrol | | | 4 g | | | | |
| UHA1028 | | | | 4 g | 2 g | 1 g | 0.5 g |

In Table 2, the amount of each component is represented as g per kg of diet.

In Table, AIN-76 (standard purified diet for mice, manufactured by CLEA Japan, Inc.), lard (manufactured by Wako Pure Chemical Industries, Ltd.), corn oil (manufactured by Wako Pure Chemical Industries, Ltd.), cholesterol (manufactured by Wako Pure Chemical Industries, Ltd.), and resveratrol (manufactured by Tokyo Chemical Industry Co., Ltd.) were used.

Control groups are the normal diet group, the high-fat diet group, and the 0.4% resveratrol intake group. The test diets fed to the control groups are, a normal diet, a high-fat diet, and a high-fat diet containing resveratrol but containing no test substance respectively.

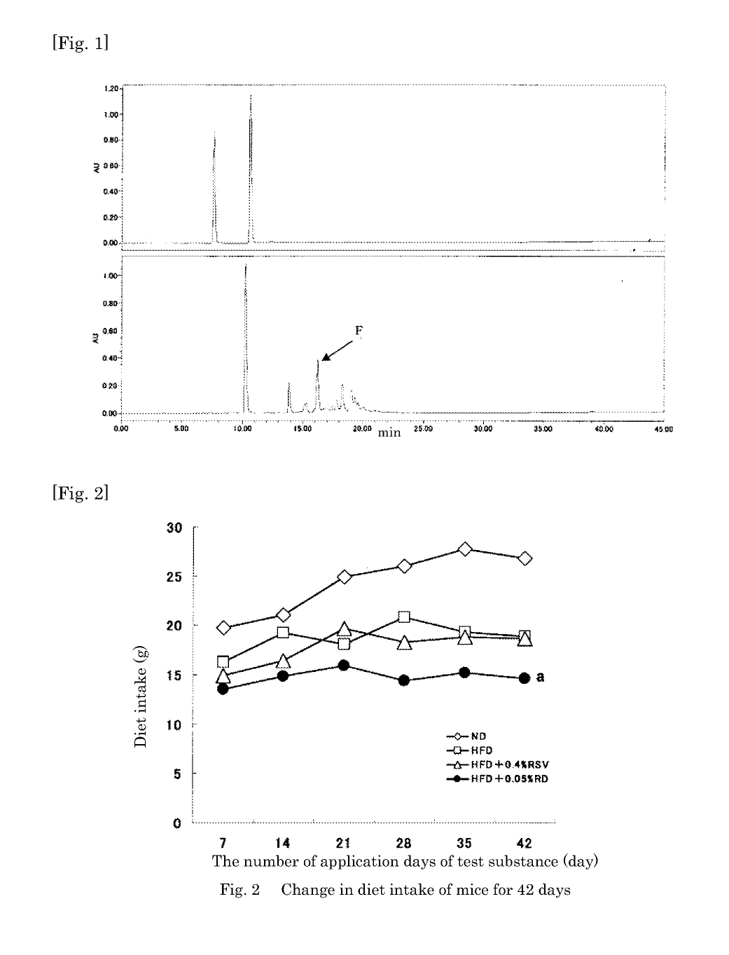

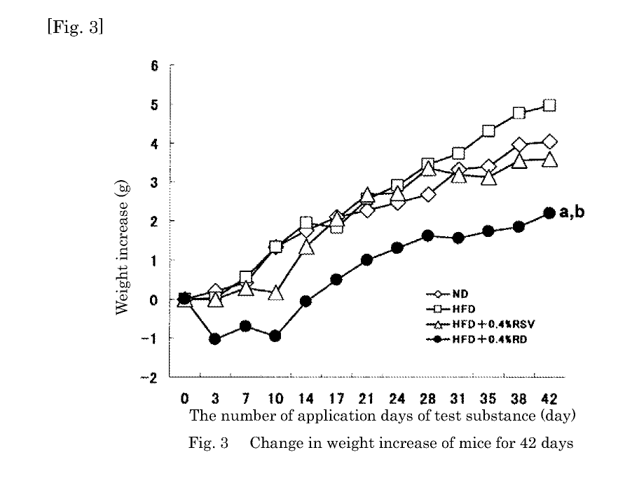

The changes in diet intake of the normal diet group, the high-fat diet group, the 0.4% resveratrol intake group, and the 0.05% UHA1028 intake group after the diet was changed to the test diet are shown in FIG. 2, and the changes in weight increase of the normal diet group, the high-fat diet group, the 0.4% resveratrol intake group, and the 0.4% UHA1028 intake group are shown in FIG. 3. In addition, the total diet intakes at the end of the test are shown in Table 3. In the UHA1028 intake group, the diet intake was significantly lower than those of the other groups (Table 3). In particular, with the 0.05% UHA1028-containing diet, it was observed that the diet intake was significantly reduced as compared with the resveratrol intake group (FIG. 2). The weight increase was also significantly suppressed as compared with the resveratrol intake group (FIG. 3).

The anatomy of the mice in the four UHA1028 intake groups at the end of the test indicated that no contraction or hypertrophy of organs was observed in each mouse. It is thus revealed that the UHA1028 has antifeeding activity and is safe even if the UHA1028 is taken for a long period of time.

TABLE 3
Total diet intake during test period

| | | Total diet |
|---|---|---|
| | Amount of test | intake |
| | substance | (g) |
| Normal diet group | — | 146.4 |
| High-fat diet group | — | 112.6 |
| Resveratrol intake group | 0.4% | 106.7 |
| UHA1028 intake group | 0.4% | 97.5 |
| | 0.2% | 99.9 |
| | 0.1% | 87.1 |
| | 0.05% | 88.4 |

The above results reveal that the UHA1028 has antifeeding activity, is highly safe, and is useful as the active component of a novel, highly practical antifeeding agent that can be taken for a long period of time.

1. An antifeeding agent comprising a (hydroxystilbene compound)-(sinapinic acid) reaction product represented by Formula (1): 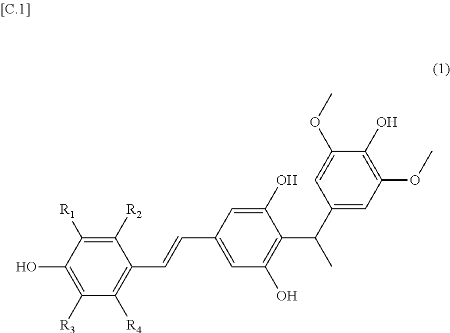
(in Formula (1), each of R₁ to R₄ is a hydrogen atom, a hydroxy group, a saturated or unsaturated, straight or branched chain alkoxy group having 1 to 10 carbon atoms, or a saturated or unsaturated, straight or branched chain alkyl group having 1 to 10 carbon atoms; and R₁ to R₄ are optionally the same as or different from each other)
or a pharmaceutically acceptable salt thereof.

2. The antifeeding agent according to claim 1, wherein R₁ to R₄ are a hydrogen atom in Formula (1).
